(19) United States
(12) Patent Application Publication
(10) Pub. No.: US 2024/0092861 A1
(43) Pub. Date: Mar. 21, 2024

(54) CD93 FUNCTIONAL DOMAINS FOR USE IN TREATING OSTEOPOROSIS

(71) Applicants: Chao-Han LAI, Tainan (TW); Jwu-Lai YEH, Tainan (TW); Hua-Lin WU, Taipei (TW); Shang-En HUANG, Kaohsiung (TW)

(72) Inventors: Chao-Han LAI, Tainan (TW); Jwu-Lai YEH, Tainan (TW); Hua-Lin WU, Taipei (TW); Shang-En HUANG, Kaohsiung (TW)

(21) Appl. No.: 18/447,229

(22) Filed: Aug. 9, 2023

Related U.S. Application Data
(60) Provisional application No. 63/407,595, filed on Sep. 16, 2022.

(51) Int. Cl. C07K 14/705; A61P 19/10

(52) U.S. Cl. CPC C07K 14/7056; A61P 19/10; C07K 2319/03

(57) ABSTRACT

CD93 functional domains for use in treating osteoporosis. A method of alleviating, reducing, suppressing, and/or treating an osteoclast-related bone disease is disclosed. The method comprises administering a therapeutically effective amount of an isolated recombinant protein comprising an amino acid sequence that is at least 80% identical to human Cluster of Differentiation 93 protein domain 1 to a subject in need thereof, the recombinant protein lacking amino acid residues 1 to 21, transmembrane and cytoplasmic domains of the human CD93 (SEQ ID NO: 3) and having a total length of no more than 559 amino acid residues. In one embodiment, the osteoclast-related bone disease is at least one selected from the group consisting of osteoporosis, postmenopausal osteoporosis, osteopenia, bone loss, inflammatory bone loss, and any combination thereof. In another embodiment, the recombinant protein comprises the amino acid sequence of SEQ ID NO: 1 or SEQ ID NO: 2.

### REFERENCE TO RELATED APPLICATION

The present application claims the priority to U.S. Provisional Application Ser. No. 63/407,595, filed Sep. 16, 2022, which is herein incorporated by reference in its entirety.

### REFERENCE TO AN ELECTRONIC SEQUENCE LISTING

The contents of the electronic sequence listing (10035-00007.xml; size 12 KB; and creation date Aug. 3, 2023) are herein incorporated by reference in its entirety.

### FIELD OF THE INVENTION

The present invention relates generally to treating bone loss, and more specifically to use of a truncated recombinant soluble CD93 (sCD93) protein for treating osteoclast-related bone disease.

### BACKGROUND OF THE INVENTION

Osteoporosis has become a growing medical and socioeconomic problem associated with significant morbidity and mortality, particularly in elderly or in post-menopausal women. Postmenopausal and age-related bone loss is due not just to accelerated bone resorption, but also to decreased bone formation (Clarket et al (2010) Radiol Clin North Am. 48(3): 483-495). Liang et al (Cellular & Molecular Biology Letters (2022) 27:72, pages 1-26) disclose that the underlying molecular mechanisms of osteoporosis are believed to be due to the increased activity of osteoclasts, decreased activity of osteoblasts, or both, which leads to an imbalance in the bone remodeling process with accelerated bone resorption and attenuated bone formation. Bone loss has also been implicated in inflammatory disorders. Hardy et al (J Endocrinol 2009; 201(3): 309-20) disclose that chronic inflammatory diseases of almost any cause are associated with bone loss. Bone loss is due to direct effects of inflammation, poor nutrition, reduced lean body mass, immobility and the effects of treatments, especially glucocorticoids. These mechanisms are complex and interrelated but are ultimately mediated through effects on the bone remodeling cycle. Inflammatory disease can increase bone resorption, decrease bone formation but most commonly impacts on both processes resulting in an uncoupling of bone formation from resorption in favor of excess resorption.

CD93 is a type 1 transmembrane glycoprotein encoded by the CD93 gene localized on chromosome 20, position p11.21 in humans. CD93 consists of a C-type lectin-like domain (CTLD), five epidermal growth factor-like domains, a mucin domain, a single transmembrane domain, and an intracellular domain. The extracellular portion of CD93 can be secreted as a soluble form (sCD93) under inflammatory conditions. Soluble CD93 (sCD93) has been implicated in various diseases associated with inflammation. Bohlson et al (J Immunol. 2005; 175:1239-47) disclose that the inflammatory mediators TNF-alpha and LPS stimulated ectodomain cleavage of CD93 from monocytes. Greenlee et al (Inflamm Res. 2009; 58:909-19) disclose that inflammation triggered release of sCD93 in vivo, in which the inflammatory macrophage was identified as a source of sCD93. Lee et al (J Clin Med 2020 May 8;9(5):1394) disclose sCD93 in type 2 diabetes as a biomarker associated with diabetic nephropathy, which is a well-known inflammatory disease. Park et al (Sci Rep. 2020; 10:323) disclose that CD93 has potential role to predict asthma in humans and the level of sCD93 significantly increased after house dust mite (HDM) stimulation via inflammatory cytokines IL-6 and thymic stromal lymphopoietin (TSLP) in vitro and in vivo. Sigari et al (Allergy Asthma Immunol Res. 2016; 8:461-5) disclose sCD93 as a novel biomarker in asthma exacerbation, in which airway inflammation is a key part of lower airway response. Malarstig et al (J Intern Med. 2011 September;270(3):229-36) disclose the concentration of sCD93 in plasma is a potential biomarker for coronary artery disease, in which inflammation is known to play a crucial role. Youn et al disclose that circulating levels of sCD93 are elevated in patients with acute myocardial infarction and their levels were associated with adverse clinical outcomes. Whether other proinflammatory molecules can interact with CTLD or other functional domains of sCD93 remains to be investigated.

Therefore, a heretofore unaddressed need exists in the art to address the deficiencies and inadequacies related to sCD93, especially in connection with pharmacological intervention of bone loss.

### SUMMARY OF THE INVENTION

In one aspect, the invention relates to a method of alleviating, reducing, suppressing, and/or treating an osteoclast-related bone disease, comprising: administering an isolated recombinant protein comprising an amino acid sequence that is at least 80% identical to human Cluster of Differentiation 93 protein domain 1 to a subject in need thereof, the recombinant protein lacking amino acid residues 1 to 21, transmembrane and cytoplasmic domains of the human CD93 (SEQ ID NO: 3) and having a total length of no more than 559 amino acid residues.

The osteoclast-related bone disease may be one selected from the group consisting of osteoporosis, postmenopausal osteoporosis, osteopenia, bone loss, inflammation-induced bone loss, and any combination thereof.

In another aspect, the invention relates to a method for treating, reducing the likelihood of, and/or protecting from reducing bone volume, and bone mineral density in a subject in need thereof, comprising: administering to the subject in need thereof an isolated recombinant protein comprising an amino acid sequence that is at least 80% identical to human Cluster of Differentiation 93 (CD93) protein domain 1, the recombinant protein lacking amino acid residues 1 to 21, transmembrane and cytoplasmic domains of the human CD93 (SEQ ID NO: 3) and having a total length of no more than 559 amino acid residues.

Further in another aspect, the invention relates to a method of treating and/or reducing the likelihood of a bone loss disease, comprising: administering to a subject in need thereof an isolated recombinant protein comprising an amino acid sequence that is at least 80% identical to human Cluster of Differentiation 93 protein domain 1, the recombinant protein lacking amino acid residues 1 to 21, transmembrane and cytoplasmic domains of the human CD93 (SEQ ID NO: 3) and having a total length of no more than 559 amino acid residues.

### BRIEF DESCRIPTION OF THE DRAWINGS

FIG. 1 shows that treatment with soluble rCD93 alleviated OVX-induced osteoporosis. a-c Representative μ-CT scanning images of the trabecular bones and quantification of trabecular bone volume/total bone volume (BV/TV) and BMD (n=12 per group). d-f Representative microscopic images of TRAP-stained histological sections of the trabecular bone and quantification of Oc.s/BS and ES/BS. g Serum CTx-1 by ELISA (n=6). ***P<0.001 compared to sham group. #P<0.05, ##P<0.01, ###P<0.001 compared to OVX-PBS group. n.s. P>0.05 compared to OVX-D123 group. Dashed lines indicate the cross sections. Blue arrows indicate TRAP-positive osteoclasts (n=6). All scale bars represent 50 μm. D123 indicates rCD93D123. D1 indicates rCD93D1. Data are represented as mean values±SEM and comparative statistical analyses were done by one-way ANOVA followed by multiple comparisons.

### DETAILED DESCRIPTION OF THE INVENTION

### Definitions

Unless otherwise defined, all technical and scientific terms used herein have the same meaning as commonly understood by one of ordinary skill in the art to which this invention pertains. In the case of conflict, the present document, including definitions will control.

As used herein, “around”“about” or “approximately” shall generally mean within 20 percent, preferably within 10 percent, and more preferably within 5 percent of a given value or range. Numerical quantities given herein are approximate, meaning that the term “around”“about” or “approximately” can be inferred if not expressly stated.

As used herein, when a number or a range is recited, ordinary skill in the art understand it intends to encompass an appropriate, reasonable range for the particular field related to the invention.

CD93 (originally identified as C1qR_{P}) is a 126 Mᵣ (reduced) type-1 transmembrane protein with an N-terminal C-type lectin-like domain, five epidermal growth factor (EGF)-like repeats, a highly glycosylated mucin-like domain and a short cytoplasmic tail. Locations of the domains in the full-length protein CD39 with amino acid residues are as follows: SP, signal peptide, 1-21; CTLD, C-type lectin-like domain, 32-174; EGF, Epidermal growth factor-like domain, 260-468; TM, Transmembrane domain, 581-601; Cyto, Cytoplasmic domain, 602-652; PDZ, PDZ binding motif, 650-652.

Serum cross-linked C-telopeptide of type I collagen (CTX) is a marker of osteoclast activity and is used to assess the level of bone resorption.

As used herein, “osteoclast-related bone disease” refers to a bone disease in which osteoclast activity has a significant contribution.

The term “treating”or “treatment” refers to administration of an effective amount of a therapeutic agent to a subject, who has a disease, a symptom or predisposition toward such a disease, with the purpose to cure, alleviate, relieve, remedy, ameliorate, or prevent the disease, the symptoms of it, or the predispositions towards it.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

Abbreviation: CD93, Cluster of Differentiation 93; CTLD, C-type like domain; BMD, bone mineral density; CTX, Serum cross-linked C-telopeptide of type I collagen.

Sequence ID NOs.:
SEQ ID NO: 1 (rCD93D1, 156 residues in length), residues Thr²² through Gly¹⁷⁷ of full-length human CD93, is the amino acid sequence of soluble rCD93 lectin-like domain (rCD93D1);
SEQ ID NO: 2 (rCD93D123, 559 residues in length), residues Thr²² through Lys⁵⁸⁰ of full-length human CD93, is the amino acid sequence of soluble rCD93 ectodomains (rCD93D123);
SEQ ID NO: 3 (full-length CD93) is the amino acid sequence of full-length human CD93;
SEQ ID NO: 4 is forward primer for rCD93D123 and rCD93D1;
SEQ ID NO: 5 is reverse primer for CD93 ectodomain 3 (D3);
SEQ ID NO: 6 is reverse primer for soluble rCD93 lectin-like domain (D1); and
SEQ ID NO: 7 (full-length CD93 DNA), is the nucleotide sequences of full-length human CD93. The nucleotides 186˜1862 of SEQ ID NO. 7 encode soluble rCD93 containing all the ectodomains (i.e., rCD93D123, SEQ ID NO. 2, which has 559 a.a. residues in length). The nucleotides 186˜653 of SEQ ID NO. 7 encode soluble rCD93 lectin-like domain (i.e., rCD93D1, SEQ ID NO: 1, which has 156 a.a. residues in length).

The invention relates to the use of recombinant protein comprising CD93 functional domains for treating bone loss. Two soluble recombinant CD93 ectodomain proteins, one containing all the ectodomains (referred as rCD93D123) and the other containing CD93 lectin-like domain (referred as rCD93D1), were studied for their therapeutic effects in suppressing osteoporosis in ovariectomized mouse model. The results proved that the soluble recombinant proteins of CD93 had potential for use in treating osteoclast-related bone diseases such as osteoporosis.

In one aspect, the invention relates to a method of alleviating, reducing, suppressing, and/or treating an osteoclast-related bone disease, comprising: administering an isolated recombinant protein comprising an amino acid sequence that is at least 80% identical to human Cluster of Differentiation 93 protein domain 1 to a subject in need thereof, the recombinant protein lacking amino acid residues 1 to 21, transmembrane and cytoplasmic domains of the human CD93 (SEQ ID NO: 3) and having a total length of no more than 559 amino acid residues.

In one embodiment of the invention, the osteoclast-related bone disease is at least one selected from the group consisting of osteoporosis, postmenopausal osteoporosis, osteopenia, bone loss, inflammation-induced bone loss, and any combination thereof.

In another aspect, the invention relates to a method for treating, reducing the likelihood of, and/or protecting from reducing bone volume, and bone mineral density in a subject in need thereof, comprising: administering to the subject in need thereof an isolated recombinant protein comprising an amino acid sequence that is at least 80% identical to human Cluster of Differentiation 93 (CD93) protein domain 1, the recombinant protein lacking amino acid residues 1 to 21, transmembrane and cytoplasmic domains of the human CD93 (SEQ ID NO: 3) and having a total length of no more than 559 amino acid residues.

In one embodiment of the invention, osteoporosis is caused by menopause.

Further in another aspect, the invention relates to a method of treating and/or reducing the likelihood of a bone loss disease, comprising: administering to a subject in need thereof an isolated recombinant protein comprising an amino acid sequence that is at least 80% identical to human Cluster of Differentiation 93 protein domain 1, the recombinant protein lacking amino acid residues 1 to 21, transmembrane and cytoplasmic domains of the human CD93 (SEQ ID NO: 3) and having a total length of no more than 559 amino acid residues.

In one embodiment, the bone loss disease is at least one selected from the group consisting of osteoporosis, postmenopausal osteoporosis, osteopenia, inflammation-induced bone loss, and any combination thereof.

In one embodiment of the invention, the total length of the recombinant protein is no more than 156 amino acid residues.

In another embodiment, the isolated recombinant protein comprises an amino acid sequence that is at least 85% identical to the human CD93 domain 1, the recombinant protein lacking amino acid residues 1 to 21, transmembrane and cytoplasmic domains of the human CD93 (SEQ ID NO: 3) and having a total length of no more than 559 amino acid residues.

In another embodiment of the invention, the isolated recombinant protein comprises an amino acid sequence that is at least 90% identical to the human CD93 domain 1, the recombinant protein lacking amino acid residues 1 to 21, transmembrane and cytoplasmic domains of the human CD93 (SEQ ID NO: 3) and having a total length of no more than 559 amino acid residues.

In another embodiment of the invention, the isolated recombinant protein comprises an amino acid sequence that is at least 95% identical to the human CD93 domain 1, the recombinant protein lacking amino acid residues 1 to 21, transmembrane and cytoplasmic domains of the human CD93 (SEQ ID NO: 3) and having a total length of no more than 559 amino acid residues.

In another embodiment of the invention, the isolated recombinant protein comprises an amino acid sequence that is identical to the human CD93 domain 1, the sequence lacking amino acid residues 1 to 21, transmembrane and cytoplasmic domains of the human CD93 (SEQ ID NO: 3) and having a total length of no more than 559 amino acid residues.

In another embodiment of the invention, the recombinant protein comprises an amino acid sequence that is at least 80% identical to the amino acid sequence of SEQ ID NO: 1 or SEQ ID NO: 2, the recombinant protein lacking amino acid residues 1 to 21, transmembrane and cytoplasmic domains of the human CD93 (SEQ ID NO: 3) and having a total length of no more than 559 amino acid residues.

In another embodiment of the invention, the recombinant protein comprises an amino acid sequence that is at least 85% identical to the amino acid sequence of SEQ ID NO: 1 or SEQ ID NO: 2, the recombinant protein lacking transmembrane and cytoplasmic domains and of the human CD93 (SEQ ID NO: 3) and having a total length of no more than 559 amino acid residues.

In another embodiment of the invention, the recombinant protein comprises an amino acid sequence that is at least 90% identical to the amino acid sequence of SEQ ID NO: 1 or SEQ ID NO: 2, the recombinant protein lacking amino acid residues 1 to 21 of the human CD93 (SEQ ID NO: 3), transmembrane and cytoplasmic domains and having a total length of no more than 559 amino acid residues.

In another embodiment of the invention, the recombinant protein comprises an amino acid sequence that is at least 95% identical to the amino acid sequence of SEQ ID NO: 1 or SEQ ID NO: 2, the recombinant protein lacking amino acid residues 1 to 21 of the human CD93 (SEQ ID NO: 3), transmembrane and cytoplasmic domains and having a total length of no more than 559 amino acid residues.

In another embodiment of the invention, the recombinant protein comprises the amino acid sequence of SEQ ID NO: 1 or SEQ ID NO: 2, the recombinant protein lacking amino acid residues 1 to 21, transmembrane and cytoplasmic domains of the human CD93 (SEQ ID NO: 3) and having a total length of no more than 559 amino acid residues.

Further in another embodiment of the invention, the recombinant protein consists essentially of the amino acid sequence of SEQ ID NO: 1 or SEQ ID NO: 2.

Yet in another embodiment of the invention, the recombinant protein consists of the amino acid sequence of SEQ ID NO: 1 or SEQ ID NO: 2.

### EXAMPLES

### Materials and Methods

Expression and Purification of rCD93D123 and rCD93D1 Protein.

A pCR3 vector (Invitrogen, Carlsbad, CA) was used for the construction of the recombinant proteins rCD93D123 (SEQ ID NO: 2) and rCD93D1 (SEQ ID NO: 1) in a mammalian HEK293 expression system. Briefly, a DNA fragment encoding the rCD93D123 (SEQ ID NO: 2), which is from (residues Thr²² to Lys⁵⁸⁰ of a full-length CD93 (SEQ ID NO: 3) was amplified by using rCD93D123 forward (5′-CCCAAGCTTATGGCCACCTCCATGGGCCTG-3′SEQ ID NO: 4) and D3 reverse primers (5′-AATTCTAGATACTTTTGCCCGTCAGTGCCA-3′SEQ ID NO: 5). A rCD93D1 fragment (SEQ ID NO. 1; residues Thr²² through Gly¹⁷⁷) was amplified by using rCD93D1 forward (5′-CCCAAGCTTATGGCCACCTCCATGGGCCTG-3′SEQ ID NO: 4) and D1 reverse primers (5′-AATTCTAGATAGTTACTTCCGGGGGAGCCT-3′SEQ ID NO: 6). The amplified DNA fragments were constructed into c-Myc as a tag for protein detection to generate rCD93. Conditioned medium containing secreted rCD93 was applied to a nickel chelating Sepharose column (Amersham Pharmacia Biotech, Piscataway, NJ). The rCD93-containing fractions were collected by gradient elution with 300 mM imidazole (Sigma-Aldrich, MO, USA). The purified rCD93D123 and rCD93D1 were examined by SDS-PAGE with coomassie blue staining and assessed by western blotting using anti-c-Myc antibody (Santa Cruz, CA, USA).

### OVX-Induced Osteoporosis Model

The female C57BL/6J mice (The Jackson Laboratory, Bar Harbor, ME) aged 8 weeks were used and maintained in a pathogen-free animal facility. The OVX-induced bone loss model was generated by bilateral ovariectomy under isoflurane anesthesia. To evaluate the effectiveness of soluble rCD93 treatment in OVX-induced bone loss, rCD93D123 (intraperitoneal injection with rCD93D123 [0.6 mg/kg] in 0.1 mL PBS once a day every 3 days since the following day after OVX), or a mole-equivalent dosage of rCD93D1 ((intraperitoneal injection with rCD93D1 [0.2 mg/kg] in 0.1 mL PBS once a day every 3 days since the following day after OVX), or the same volume of PBS. At 8 weeks, the mice were sacrificed for μ-CT analyses and harvest of lower limbs. Bone samples from each group were imaged using a SkyScan-1076 Micro-CT System (Skyscan, Kontich, Belgium). The μ-CT scanner was operated at 45 kV, 220 μA, 0.4μ rotation step, 0.5 mm aluminum filter, and a scan resolution of 18 μm/pixel. The following 3D parameters, including bone mineral density (BMD), total bone volume (TV), trabecular bone volume (BV), and trabecular bone volume fraction (BV/TV), were evaluated using the software CT Analyser (Bruker, Kontich, Belgium). These experiments were approved by the Animal Care and Use Committee of Kaohsiung Medical University Animal Center and conformed to the Guide for the Care and Use of Laboratory Animals published by the National Institutes of Health (NIH Publication #85-23, revised 1996). The extracted lower limbs from the OVX-induced osteoporosis model were fixed in 10% formalin, and the samples were dehydrated in 10% EDTA for 14 days and embedded in paraffin. The paraffin sections were stained with TRAP staining (387A, Sigma-Aldrich) according to the manufacturers' protocols. Mouse CTx-1 (CEA665Mu, Cloud-Clone Corp) in the serum was analyzed using commercially available ELISA kits according to the manufacturers' protocols.

Treatment with Soluble rCD93 Alleviates OVX-Induced Osteoporosis.

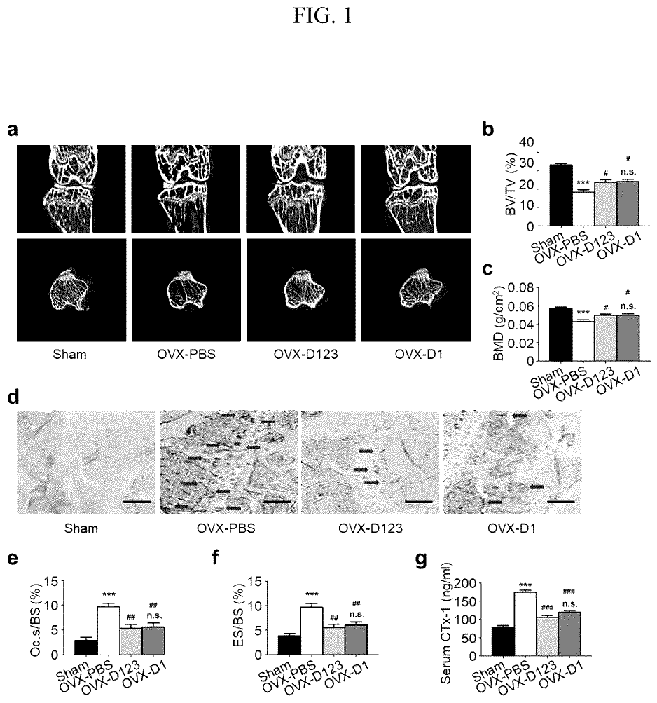

The protective effects of rCD93D123 and rCD93D1 treatment on bone loss were evaluated using the OVX-induced model. Micro-computerized tomography scans showed that the reduced bone volume fraction (BV/TV) and bone mineral density (BMD) in PBS-treated mice were prevented in rCD93D123 and rCD93D1 treated mice, and the levels of protection in rCD93D123 and rCD93D1 treated mice were not significantly different (FIG. 1a-c).

TRAP staining of histological sections showed that the increased ratios of osteoclast-covered surface (Oc.s/BS) and eroded surface (ES/BS) per bone surface in PBS-treated mice were alleviated in rCD93D123 and rCD93D1 treated mice, and the levels of alleviation were not significantly different in rCD93D123 and rCD93D1 treated mice (FIG. 1d-f). It can be concluded that the osteoclasts (population or activity, or both population and activity) appeared to be suppressed in ovariectomized mice treated with rCD93 D123 or rCD93D1 (e-f).

Consistent with the histological findings, the increased serum level of the bone resorption marker CTx-1 in PBS-treated mice was reduced in rCD93D123 and rCD93D1 treated mice, and the CTx-1 levels in rCD93D123and rCD93D1-treated mice were not significantly different (FIG. 1g). It can be concluded that the osteoclast activity was inhibited in ovariectomized mice treated with rCD93 D123 or rCD93D1 (FIG. 1g).

Taken together, these results revealed that treatment with rCD93D123 or rCD93D1 could attenuate bone resorption and osteoporosis in the OVX-induced model. Our results support rCD93D123 and rCD93D1 as potential agents for novel anti-osteoclast-related diseases such as osteoporosis.

All references cited and discussed in this specification are incorporated herein by reference in their entireties and to the same extent as if each reference was individually incorporated by reference.

What is claimed is:

1. A method of alleviating, reducing, suppressing, and/or treating an osteoclast-related bone disease, comprising:
administering an isolated recombinant protein comprising an amino acid sequence that is at least 80% identical to human Cluster of Differentiation 93 protein domain 1 to a subject in need thereof, the recombinant protein lacking amino acid residues 1 to 21, transmembrane and cytoplasmic domains of the human CD93 (SEQ ID NO: 3) and having a total length of no more than 559 amino acid residues.

2. The method of claim 1, wherein the total length of the isolated recombinant protein is 559 amino acid residues.

3. The method of claim 1, wherein the total length of the recombinant protein is no more than 156 amino acid residues.

4. The method of claim 1, wherein the isolated recombinant protein comprises an amino acid sequence that is at least 90% identical to the human CD93 domain 1, the recombinant protein lacking the amino acid residues 1 to 21, the transmembrane and cytoplasmic domains of the human CD93 (SEQ ID NO: 3) and having a total length of no more than 559 amino acid residues.

5. The method of claim 1, wherein the isolated recombinant protein comprises an amino acid sequence that is identical to the human CD93 domain 1, the sequence lacking the amino acid residues 1 to 21, the transmembrane and cytoplasmic domains of the human CD93 (SEQ ID NO: 3) and having a total length of no more than 559 amino acid residues.

6. The method of claim 1, wherein the recombinant protein comprises an amino acid sequence that is at least 80% identical to the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

7. The method of claim 1, wherein the recombinant protein comprises an amino acid sequence that is at least 85% identical to the amino acid sequence of SEQ ID NO: 1 or SEQ ID NO: 2, the recombinant protein lacking the transmembrane and cytoplasmic domains and of the human CD93 (SEQ ID NO: 3) and having a total length of no more than 559 amino acid residues.

8. The method of claim 1, wherein the recombinant protein comprises an amino acid sequence that is at least 90% identical to the amino acid sequence of SEQ ID NO: 1 or SEQ ID NO: 2, the recombinant protein lacking the amino acid residues 1 to 21 of the human CD93 (SEQ ID NO: 3), the transmembrane and cytoplasmic domains and having a total length of no more than 559 amino acid residues.

9. The method of claim 1, wherein the recombinant protein comprises the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

10. The method of claim 1, wherein the osteoclast-related bone disease is at least one selected from the group consisting of osteoporosis, postmenopausal osteoporosis, osteopenia, bone loss, inflammatory bone loss, and any combination thereof.

11. A method for treating, reducing the likelihood of, and/or protecting from reducing bone volume, and bone mineral density in a subject in need thereof, comprising:
administering to the subject in need thereof an isolated recombinant protein comprising an amino acid sequence that is at least 80% identical to human Cluster of Differentiation 93 (CD93) protein domain 1, the recombinant protein lacking amino acid residues 1 to 21, transmembrane and cytoplasmic domains of the human CD93 (SEQ ID NO: 3) and having a total length of no more than 559 amino acid residues.

12. The method of claim 11, wherein the isolated recombinant protein comprises an amino acid sequence that is at least 90% identical to the human CD93 domain 1, the recombinant protein lacking the amino acid residues 1 to 21, the transmembrane and cytoplasmic domains of the human CD93 (SEQ ID NO: 3) and having a total length of no more than 559 amino acid residues.

13. The method of claim 11, wherein the isolated recombinant protein comprises an amino acid sequence identical to the human CD93 domain 1, the sequence lacking amino acid residues 1 to 21, transmembrane and cytoplasmic domains of the human CD93 (SEQ ID NO: 3) and having a total length of no more than 559 amino acid residues.

14. The method of claim 11, wherein the recombinant protein comprises an amino acid sequence that is at least 80% identical to the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

15. The method of claim 11, wherein the recombinant protein comprises an amino acid sequence that is at least 90% identical to the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

16. The method of claim 11, wherein the recombinant protein comprises the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

17. A method of treating and/or reducing the likelihood of a bone loss disease, comprising:
administering to a subject in need thereof an isolated recombinant protein comprising an amino acid sequence that is at least 80% identical to human Cluster of Differentiation 93 protein domain 1, the recombinant protein lacking amino acid residues 1 to 21, transmembrane and cytoplasmic domains of the human CD93 (SEQ ID NO: 3) and having a total length of no more than 559 amino acid residues.

18. The method of claim 17, wherein the recombinant protein comprises an amino acid sequence that is at least 80% identical to the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

19. The method of claim 17, wherein the recombinant protein comprises an amino acid sequence that is at least 90% identical to the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.

20. The method of claim 17, wherein the recombinant protein comprises the amino acid sequence of SEQ ID NO: 1 or SEQ ID NO: 2, the recombinant protein lacking the amino acid residues 1 to 21, the transmembrane and cytoplasmic domains of the human CD93 (SEQ ID NO: 3) and having a total length of no more than 559 amino acid residues.


SEQUENCE LISTING (7 sequences)

SEQ ID NO: 1 - protein, 156 aa, Homo sapiens
TGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEG

SEQ ID NO: 2 - protein, 559 aa, Homo sapiens
TGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPNRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSSGVWREPSIHHATAASGPQEPAGGDSSVATQNNDGTDGQK

SEQ ID NO: 3 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPNRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSSGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 4 - DNA, 30 bp, synthetic construct
  source 1..30: mol_type = other DNA
cccaagcttatggccacctccatgggcctg

SEQ ID NO: 5 - DNA, 30 bp, synthetic construct
  source 1..30: mol_type = other DNA
aattctagatacttttgcccgtcagtgcca

SEQ ID NO: 6 - DNA, 30 bp, synthetic construct
  source 1..30: mol_type = other DNA
aattctagatagttacttccgggggagcct

SEQ ID NO: 7 - DNA, 3337 bp, Homo sapiens
  source 1..3337: mol_type = genomic DNA
ctgcgccggagtggctgcagctcacccctcagctccccttggggcccagctgggagccgagatagaagctcctgtcgccgctgggcttctcgcctcccgcagagggccacacagagaccgggatggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgccctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgcgctgcaacagccagtcccacaaggggccccgagggcacccccaaggctacacccaccacaagtagaccttcgctgtcatctgacgcccccatcacatctgccccactcaagatgctggcccccagtgggtcctcaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgctgaaagtgaggtggccctagagacactagagtcaccagccaccatcctcagagctttgaactccccattccaaaggggcacccacatttttttgaaagactggactggaatcttagcaaacaattgtaagtctcctccttaaaggccccttggaacatgcaggtattttctacgggtgtttgatgttcctgaagtggaagctgtgtgttggcgtgccacggtggggatttcgtgactctataatgattgttactccccctcccttttcaaattccaatgtgaccaattccggatcagggtgtgaggaggctggggctaaggggctcccctgaatatcttctctgctcacttccaccatctaagaggaaaaggtgagttgctcatgctgattaggattgaaatgatttgtttctcttcctaggatgaaaactaaatcaattaattattcaattaggtaagaagatctggttttttggtcaaagggaacatgttcggactggaaacatttctttacatttgcattcctccatttcgccagcacaagtcttgctaaatgtgatactgttgacatcctccagaatggccagaagtgcaattaacctcttaggtggcaaggaggcaggaagtgcctctttagttcttacatttctaatagccttgggtttatttgcaaaggaagcttgaaaaatatgagaaaagttgcttgaagtgcattacaggtgtttgtgaagtcacataatctacggggctagggcgagagaggccagggatttgttcacagatacttgaattaattcatccaaatgtactgaggttaccacacacttgactacggatgtgatcaacactaacaaggaaacaaattcaaggacaacctgtctttgagccagggcaggcctcagacaccctgcctgtggccccgcctccacttcatcctgcccggaatgccagtgctccgagctcagacagaggaagccctgcagaaagttccatcaggctgtttcctaaaggatgtgtgaacgggagatgatgcactgtgttttgaaagttgtcattttaaagcattttagcacagttcatagtccacagttgatgcagcatcctgagattttaaatcctgaagtgtgggtggcgcacacaccaagtagggagctagtcaggcagtttgcttaaggaacttttgttctctgtctcttttccttaaaattgggggtaaggagggaaggaagagggaaagagatgactaactaaaatcattt